(19) United States
(12) Patent Application Publication
(10) Pub. No.: US 2023/0047207 A1
(43) Pub. Date: Feb. 16, 2023

(54) Self-Contained Biological Indicator with Salt Compound

(71) Applicant: 3M INNOVATIVE PROPERTIES COMPANY, St. Paul, MN (US)

(72) Inventors: Francois Ahimou, Woodbury, MN (US); Kelvin J. Witcher, Hudson, WI (US); Naiyong Jing, St. Paul, MN (US); Tonya D. Bonilla, Woodbury, MN (US); G. Marco Bommarito, Stilwater, MN (US)

(21) Appl. No.: 17/758,585

(22) PCT Filed: Jan. 14, 2021
(86) PCT No.: PCT/IB2021/050263

§ 371 Date: Jul. 11, 2022

Related U.S. Application Data
(60) Provisional application No. 62/964,369, filed on Jan. 22, 2020.

(51) Int. Cl. C12M 1/12; C12Q 1/22; A61L 2/28

(52) U.S. Cl. CPC C12M 37/06; C12Q 1/22; A61L 2/28

(57) ABSTRACT

The present disclosure is directed to self-contained biological indicators wherein a single type of indicator is capable of being used for various sterilization conditions, including sterilization with steam, hydrogen peroxide, and/or ethylene oxide. In some embodiments, a single type of biological indicator is capable of being used for different steam sterilization conditions having varied temperatures and sterilization cycles.

The present disclosure is directed to self-contained biological indicators that can be used under various sterilization conditions, including sterilization with steam, ethylene oxide, and/or other sterilants including, in particular, hydrogen peroxide vapor. In some embodiments, a single type of biological indicator is capable of being used under different steam sterilization conditions having vaned temperatures and sterilization cycles.

### BACKGROUND

The sterilization of equipment, instruments, and other devices is critical in the health care industry. For example, hospitals and other medical institutions frequently sterilize medical instruments and equipment used in treating patients. The particular type of sterilization cycle used to sterilize such equipment can vary based on the particular equipment or devices being sterilized and based on the particular preference of the entity performing the sterilization cycle. However, all such sterilization cycles or processes are typically designed to kill living organisms which might otherwise contaminate the equipment or devices being sterilized.

Various sterilization methods use different cycles or techniques for sterilization. For instance, sterilization may include the administration of steam, dry heat, chemicals (e.g., ethylene oxide), vapor phase hydrogen peroxide (VPHP), or radiation, to the equipment or devices being sterilized. Steam sterilization is typically efficacious when the equipment being sterilized is heat resistant at high temperatures because the items are exposed to steam having a temperature generally in a range of 121-135° C. The period of exposure to steam depends on the sterilization temperature. For example, equipment or instruments can be exposed to the steam sterilization process under varying temperature and time standards, such as, for example, approximately three minutes at 132° C. or up to 30 minutes or more at 121° C. Sterilization modalities based on vapor phase hydrogen peroxide (VPHP) include those generally designated as vaporized hydrogen peroxide (VHP) sterilization as well as modalities that include a hydrogen peroxide plasma and are generally designated as hydrogen peroxide gas plasma (HPGP) sterilization.

Other types of sterilization involve exposing the devices or instruments to chemical agents. A common chemical sterilant used for low-temperature sterilization is ethylene oxide gas. Typically, for ethylene oxide sterilization, the devices being sterilized are exposed to the ethylene oxide gas for a period ranging from one hour at 55° C. to approximately four hours at 38° C. Dry heat sterilization typically involves exposing the devices being sterilized to temperatures in a range of approximately 180° C., or higher, for at least two hours. In many medical applications, the efficacy of the sterilization cycle is critical.

Biological indicators are commonly used to evaluate and validate the effectiveness of a sterilization process in a variety of settings. In general, viable but relatively highly-resistant spores of thermophilic organisms are subjected to the sterilization conditions along with any devices or instruments to be sterilized. In general, the test microorganisms are more resistant to the sterilization cycle than most other organisms that would be present by natural contamination. Applicants have used spores of microorganisms capable of producing an enzyme that catalyzes the reaction of a non-fluorescent substrate to a fluorescent product that can be detected to indicate the presence of surviving spores.

Typically, after completion of the sterilization cycle, the spores are incubated in nutrient medium to determine whether any of the test organisms survived the sterilization procedure. In the conventional biological indicators, growth of a detectable number of organisms can take 24 hours or more for a pH color change indicator.

The biological indicator is then examined to determine whether such growth has taken place. Applicants use rapid readout technology based on a fluorescent response during incubation in the growth medium due to the release of an enzyme during the spore germination and outgrowth. When media comes in contact with viable spores, the spore-associated enzyme interacts with fluorogenic substrate contained in the media. The interaction of the enzyme and substrate results in the cleavage of the substrate to produce a fluorescently-detected compound. An analysis of the fluorescence intensity due to the fluorescent product correlated with other parameters serves to determine whether the sterilization process was successful.

In general, biological indicators are designed for specific cycles and Applicants know of no biological indicator that can be used under all common commercially-available sterilization cycles. The present disclosure is directed to biological indicators that can be used for one or more of the commercial steam sterilization cycles.

### SUMMARY

In one aspect, the present disclosure provides self-contained biological indicators. The self-contained biological indicators can be used for determining the efficacy of a given sterilization cycle and to items comprising those biological indicators. In other embodiments, the same biological indicator is capable of determining the efficacy of most or all steam sterilization cycles. In some embodiments, the biological indicators can determine the efficacy of a sterilization cycle in less than 60 minutes.

In one aspect, the present disclosure provides a self-contained biological indicator. The self-contained biological indicator can comprise a housing. The housing can contain a plurality of test microorganisms comprising and/or capable of producing an enzyme capable of catalyzing a cleavage of an enzyme substrate, a nutrient composition, the enzyme substrate, a container containing a liquid composition, and an effective amount of a salt compound. The nutrient composition facilitates germination and/or outgrowth of the test microorganisms. the container is adapted to allow selective fluid communication between the liquid composition and the test microorganisms. The effective amount of the salt compound, when dissolved in the liquid composition, is present at a concentration of at least 0.5 mM and up to 50 mM of the salt compound in the liquid composition; with the proviso that when the concentration equals 10 mM, the salt compound is not potassium phosphate. The cleavage of the enzyme substrate by the enzyme produces a fluorescently detectable compound.

In any embodiment, salt compound can be a salt of any ion selected from the group consisting of acetate; borate; citrate; carbonate; bicarbonate; phosphate; hydrogen phosphate; dihydrogen phosphate; chloride; sulfate; N,N-bis(2-hydroxyethyl)-2-aminoethanesulfonate; N,N-bis(2-hydroxyethyl)glycine; 3-(cyclohexylamino)-2-hydroxy-1-propanesulfonic acid; N-cyclohexyl-2-aminoethanesulfonate; imidazolium; 2-(N-morpholino)ethanesulfonate; 3-(N-morpholino)propanesulfonic acid; tricine, 2-amino-2-(hydroxymethyl)propane-1,3-diol; and a combination of any two or more of the foregoing salts. In any of the above embodiments, the enzyme can be selected from the group consisting of α-glucosidase, α-galactosidase, lipase, esterase, acid phosphatase, alkaline phosphatase, protease, aminopeptidase, chymotrypsin, β-glucosidase, β-galactosidase, α-glucoronidase, β-glucoronidase, phosphohydrolase, calpain, α-mannosidase, β-mannosidase, α-L-fucosidase, leucine aminopeptidase, α-L-arabinofuranosidase, cysteine aminopeptidase, valine aminopeptidase, β-xylosidase, glucanase, cellobiosidase, cellulase, α-arabinosidase, glycanase, sulfatase, butyrase, glycosidase, arabinosidase, and a combination of any two or more of the foregoing enzymes. In any of the above embodiments, the enzyme substrate comprises a derivative of 4-methylumbelliferone or a derivative of 7-amino-4-methylcoumarin. In any of the above embodiments, the enzyme comprises α-D-glucosidase, wherein the enzyme substrate comprises 4-methylumbelliferyl-α-D-glucopyranoside.

In another aspect, the present disclosure provides a kit. In certain embodiments, the kit can comprise any of the above embodiments of the self-contained biological indicator. In certain alternative embodiments, the kit can comprise a housing, a plurality of test microorganisms comprising and/or capable of producing an enzyme capable of catalyzing a cleavage of an enzyme substrate, a nutrient composition, the enzyme substrate, a container containing a liquid composition, and an effective amount of a salt compound. The nutrient composition facilitates germination and/or outgrowth of the test microorganisms. The enzyme substrate comprises a fluorescently detectable component. The effective amount of the salt compound, when dissolved in the liquid composition, yields a concentration of at least 0.5 mM and up to 50 mM of the salt compound in the liquid composition; with the proviso that when the concentration equals 10 mM, the salt compound is not potassium phosphate. In any of the above embodiments of the kit, one or more of the nutrient composition, the enzyme substrate, the liquid composition, the salt compound, and the plurality of test microorganisms can be disposed in the housing. In any of the above embodiments of the kit, the liquid composition can be disposed in a frangible container.

In yet another aspect, the present disclosure provides a system for use in determining the efficacy of a sterilization process. The system can comprise any of the above embodiments of the self-contained biological indicator, and an automated reader. The automated reader can be configured to receive at least a portion of the biological indicator, direct a first wavelength of electromagnetic radiation into the liquid composition in the housing, and detect or measure a quantity of a second wavelength of electromagnetic radiation emitted by the fluorescent product. In any of the above embodiments of the system, the self-contained biological indicator is adapted to be used to determine efficacy of any steam sterilization process selected from the group consisting of 121° C. gravity process, 121° C. pre-vac process, 121° C. SFPP process, 132° C. gravity process, 132° C. pre-vac process, 132° C. SFPP process, 134° C. pre-vac process, 134° C. SFPP process, 135° C. gravity process, 135° C. pre-vac process, and 135° C. SFPP process. SFPP means steam flush pressure pulse and pre-vac means pre-vacuum or vacuum-assisted both are considered aspects of dynamic air removal as opposed to gravity, which is a passive air removal process.

In yet another aspect, the present disclosure provides a method for determining efficacy of a sterilization process. The method can comprise exposing a plurality of test microorganisms that are disposed in a housing to the sterilization process, wherein the plurality of test microorganisms comprises and/or is capable of producing an enzyme capable of reacting with an enzyme substrate to produce a fluorescent product. The method further can comprise, after exposing the test microorganisms to the sterilization process, bringing the plurality of test microorganisms into contact with a liquid composition. Bringing the plurality of test microorganisms into contact with the liquid composition comprises placing the test microorganisms in liquid contact with the fluorogenic enzyme substrate. after bringing the test microorganisms into contact with the liquid composition, a resulting mixture of the plurality of test microorganisms and the liquid composition comprises a nutrient composition, the enzyme substrate, and a salt compound; wherein the salt compound is present in the mixture at a concentration of at least 0.5 mM and up to 50 mM of the salt compound in the liquid composition; with the proviso that when the concentration equals 10 mM, the salt compound is not potassium phosphate. The nutrient composition facilitates germination and/or outgrowth of the test microorganisms. The method further can comprise incubating the mixture for a period of time and detecting the fluorescent product in the mixture, wherein detecting at least a threshold quantity of the fluorescent product indicates a lack of efficacy of the sterilization process.

In any of the above embodiments of the method, incubating the mixture for a period of time comprises incubating the mixture at a specified temperature. In any of the above embodiments of the method, the period of time is a specified period of time, wherein the specified period of time is less than or equal to 180 minutes, wherein detecting less than a threshold quantity of the :fluorescent product after the specified period of time indicates efficacy of the sterilization process. In any of the above embodiments of the method, detecting the fluorescent product can comprise quantifying fluorescence emitted by the fluorescent product.

All scientific and technical terms used herein have meanings commonly used in the art unless otherwise specified. The definitions provided herein are to facilitate understanding of certain terms used frequently in this application and are not meant to exclude a reasonable interpretation of those terms in the context of the present disclosure.

Unless otherwise indicated, all numbers in the description and the claims expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

The recitation of numerical ranges by endpoints includes all numbers subsumed within that range (e.g. a range from 1 to 5 includes, for instance, 1, 1.5, 2, 2.75, 3, 3.80, 4, and 5) and any range within that range.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “powerset” as used herein for a given set S having n elements refers to the mathematical definition of a powerset and all possible subsets of S, without including the empty set, but including S itself, having from 1 to n elements in every combination and is denoted as P(S). Applicants note that the mathematical definition of a powerset includes the empty set (a set having no elements. However, the definition adopted here by Applicants excludes the empty set and includes all subsets having at least one element, including the full set of n elements (S). In general, the powerset includes all subsets having “i” elements for i=1 to n−1, and the subset having all n elements (n). For instance, the powerset of a subset S having the elements a, b, and c (n=3) includes the following 7 subsets: all possible subsets having one element: {(a), (b), (c)}; all possible subsets having any possible combination two elements: {(a, b), (a, c), (b, c)}, and the subset having all 3 elements: (a, b, c).

The term “frangible” container refers to any container that can be acted upon to release its contents, for example by breaking it, puncturing it, shattering it, cutting it, etc.

The term “process challenge device,” abbreviated as “PCD,” refers to a container that may comprise a biological indicator inside and which contains an additional barrier to a sterilant to reach its contents (e.g., a biological indicator) compared to the path the sterilant would need to travel to reach the items insider the PCD (e.g., biological indicator) if the items were not inside the PCD. A PCD is also known as a “test pack” and both terms are being used interchangeably in this disclosure. A PCD is designed to simulate sterilization conditions used for instruments or other items to be sterilized and generally comprise a defined challenge to the sterilization process. In its most simply embodiment, a PCD is a sealed container that has an inlet (e.g., an orifice or puncture) for a sterilant to be able to reach the interior of the container.

The term “fluorescently-detectable compound” refers to a compound that is susceptible to detection by fluorescence, even if the compound may not be fluorescent at all times and only fluoresces when exited by energy of the proper wavelength. Examples of fluorescently-detectable compound useful in this patent application include the products of an enzymatic reaction of a substrate with a cleaving enzyme where the substrate is not fluorescently-detectable using the excitation wavelengths used to detect the enzymatic reaction product. The fluorescent detection can be carried out in solution or on a substrate. An example of such a compound is 4-methylumbelliferone (4-MU), which is the product of the enzymatic cleavage of 4-methylumbelliferyl-α-D-glucopyranoside by the enzyme α-D-glucosidase.

The term “adjacent” refers to the relative position of two elements, such as, for example, two layers, that are close to each other and may or may not be necessarily in contact with each other or that may have one or more layers separating the two elements as understood by the context in which “adjacent” appears.

The term “immediately adjacent” refers to the relative position of two elements, such as, for example, two layers, that are next to each other and in contact with each other and have no intermediate layers separating the two elements. The term “immediately adjacent,” however, encompasses situations where one or both elements (e.g., layers) have been treated with a primer, or whose surface has been modified to affect the properties thereof, such as etching, embossing, etc., or has been modified by surface treatments, such as corona or plasma treatment, etc. that may improve adhesion.

The above summary is merely intended to provide a cursory overview of the subject matter of the present disclosure and is not intended to describe each disclosed embodiment or every implementation of the present invention. The description that follows more particularly exemplifies illustrative embodiments. In several places throughout the application, guidance is provided through lists of examples, which can be used in various combinations. In each instance, the recited list serves only as a representative group and should not be interpreted as an exclusive list.

### BRIEF DESCRIPTION OF THE DRAWINGS

FIG. 1 represents a schematic view of an exemplary biological indicator of the present disclosure.

FIG. 2 represents an expanded view of an exemplary biological indicator of the present disclosure.

### DETAILED DESCRIPTION

The present disclosure provides a self-contained biological indicator (SCBI). The SCBI can be used to assess the efficacy of a sterilization process. The SCBI comprises a plurality of test microorganisms comprising and/or capable of producing an enzyme capable of catalyzing a cleavage of an enzyme substrate, a nutrient composition, the enzyme substrate, a container containing a liquid composition, and an effective amount of a salt compound. The nutrient composition facilitates germination and/or outgrowth of the test microorganisms. the container is adapted to allow selective fluid communication between the liquid composition and the test microorganisms. The effective amount of the salt compound, when dissolved in the liquid composition, is present at a concentration of at least 0.5 mM and up to 50 mM of the salt compound in the liquid composition; with the proviso that when the concentration equals 10 mM, the salt compound is not potassium phosphate. The cleavage of the enzyme substrate by the enzyme produces a fluorescently detectable compound.

It is now known that the addition of the effective amount of the salt compound can improve the detection of the enzyme that catalyzes the enzyme substrate to produce the fluorescently detectable compound (e.g., by increasing the activity of the enzyme and/or by stabilizing the fluorescent signal and/or by causing an improved correlation of the detection of the enzyme activity and the detection of growth of the test microorganisms after the biological indicator is exposed to a sterilization process).

### Self-Contained Biological Indicators

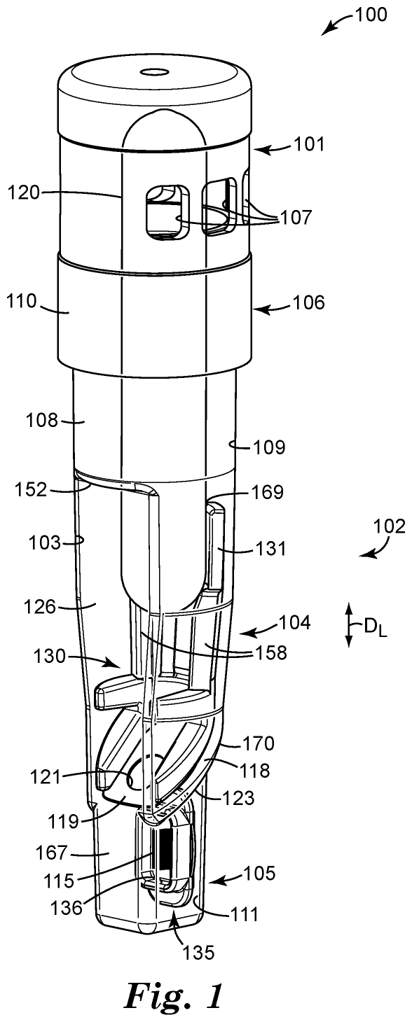

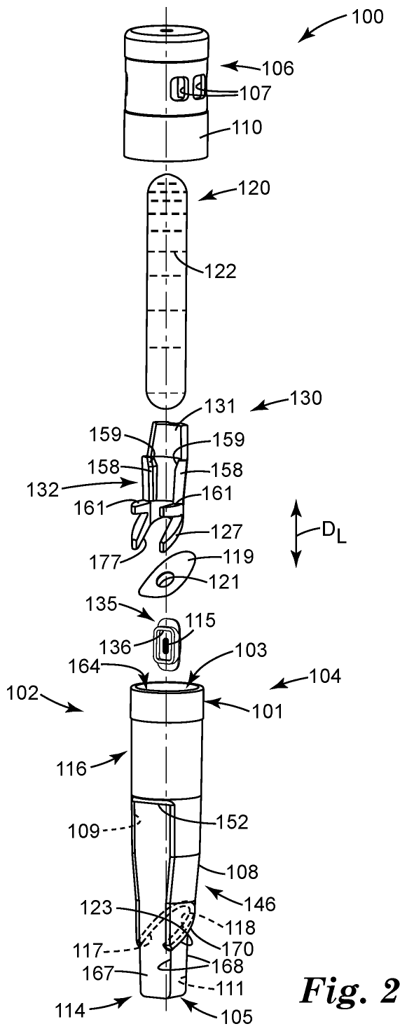

Turning now to FIGS. 1 and 2, an exemplary biological indicator 100 can include a housing 102, which can include a first portion 104 and a second portion 106 (e.g., a cap) adapted to be coupled together to provide a self-contained biological indicator. In some embodiments, the first portion 104 and second portion 106 can be formed of the same materials, and in some embodiments, the first portion 104 and the second portion 106 can be formed of different materials. The housing 102 can define a reservoir 103 of the biological indicator 100 in which other components can be positioned and into which a sterilant can be directed during a sterilization process.

The housing 102 can be defined by at least one liquid impermeable wall, such as a wall 108 of the first portion 104 and/or a wall 110 of the second portion 106. It should be understood that a one-part unitary housing 102 may also be employed or that the first and second portions 104 and 106 can take on other shapes, dimensions, or relative structures without departing from the spirit and scope of the present disclosure. Suitable materials for the housing 102 (e.g., the walls 108 and 110) can include, but are not limited to, a glass, a metal (e.g., foil), a polymer (e.g., polycarbonate (PC), polypropylene (PP), polyphenylene (PPE), polythyene, polystyrene (PS), polyester (e.g., polyethylene terephthalate (PET)), polymethyl methacrylate (PMMA or acrylic), acrylonitrile butadiene styrene (ABS), cyclo olefin polymer (COP), cyclo olefin copolymer (COC), polysulfone (PSU), polyethersulfone (PES), polyetherimide (PEI), polybutyleneterephthalate (PBT), a ceramic, a porcelain, or combinations thereof.

In some embodiments, the biological indicator 100 can further include a frangible container 120 that contains a liquid (e.g., liquid composition) 122, and which is dimensioned to be received within the biological indicator 100, for example, within at least a portion of the housing 102 (e.g., at least within the first portion 104 of the housing 102). The frangible container 120 can be formed of a variety of materials, including, but not limited to, one or more of metal (e.g., foil), a polymer (e.g., any of the polymers listed above with respect to the housing 102), glass (e.g., a glass ampoule), and combinations thereof. In some embodiments, only a portion of the container 120 is frangible, for example, the container 120 can include a frangible portion or cover (e.g., a frangible barrier, film, membrane, or the like). The frangible container 120 can have a first state in which it is intact and the liquid 122 is contained therein, and a second state in which at least a portion of the container 120 is fractured. In the second state of the container 120, the liquid 122 can be in fluid communication with the reservoir 103 of the biological indicator 100, e.g., when the container 120 is positioned in the biological indicator 100.

As shown in the illustrated embodiment in FIGS. 1 and 2, the container 120 can be held in place within the biological indicator 100 and/or fractured by an insert 130.

The first portion 104 of the housing 102 can be adapted to house a majority of the components of the biological indicator 100, and can be referred to as the “body,” or “tube,” “tubular body,” “base,” or the like. The housing 102 can include a reservoir 103 that can be defined by one or both of the first portion 104 and the second portion 106 of the housing 102. The biological indicator 100 can further include test microorganisms 115 such as spores, for example, (or a locus of test microorganisms) positioned in fluid communication with the reservoir 103. As shown in FIGS. 1-2, the second portion 106 of the housing 102 can include one or more apertures 107 to provide fluid communication between the interior of the housing 102 (e.g., the reservoir 103) and ambience. For example, the one or more apertures 107 can provide fluid communication between the test microorganisms 115 and ambience during a sterilization process and can serve as an inlet into the biological indicator 100 and as an inlet of a sterilant path 164 (described in greater detail below). In some embodiments, the second portion 106 of the housing 102 can be coupled to a first (e.g., open) end 101 of the first portion 104 of the housing 102, and the test microorganisms 115 can be positioned at a second (e.g., closed) end 105, opposite the first end 101, of the first portion 104 of the housing 102.

In some embodiments, a barrier or filter (e.g., a sterile barrier; not shown) can be positioned in the sterilant path 164 (e.g., at the inlet formed by the aperture 107) to inhibit contaminating or foreign organisms, objects or materials from entering the biological indicator 100. Such a barrier can include a gas-transmissive, microorganism-impermeable material, and can be coupled to the housing 102 by a variety of coupling means, including, but not limited to, an adhesive, a heat seal, sonic welding, or the like. Alternatively, the barrier can be coupled to the sterilant path 164 via a support structure (such as the second portion 106) that is coupled to the first portion 104 of the housing 102 (e.g., in a snap-fit engagement, a screw-fit engagement, a press-fit engagement, or a combination thereof). During exposure to a sterilant, the sterilant can pass through the barrier into the sterilant path 164 and into contact with the test microorganisms 115.

In some embodiments, as shown in the illustrated embodiments, the housing 102 can include a lower portion 114 and an upper portion 116, which can be at least partially separated by an inner wall (or partial wall) 118, ledge, partition, flange, or the like, in which can be formed an opening 117 that provides fluid communication between the lower portion 114 and the upper portion 116. In some embodiments, the lower portion 114 of the first portion 104 of the housing 102 (sometimes referred to as simply “the lower portion 114” or the “the lower portion 114 of the housing 102”) can be adapted to house the test microorganisms 115 or a locus of test microorganisms. In some embodiments, the lower portion 114 can be referred to as the “detection portion” or “detection region” of the housing 102, because at least a portion of the lower portion 114 can be interrogated for signs of test microorganisms growth. In addition, in some embodiments, the upper portion 116 of the first portion 104 of the housing 102 (sometimes referred to as “the upper portion 116” or the “the upper portion 116 of the housing 102” for simplicity) can be adapted to house at least a portion of the frangible container 120, particularly before activation.

In some embodiments, the portion of the reservoir 103 that is defined at least partially by the upper portion 116 of the housing 102 can be referred to as a first chamber (or reservoir, zone, region, or volume) 109 and the portion of the reservoir 103 that is defined at least partially by the lower portion 114 of the housing 102 can be referred to as a second chamber (or reservoir, zone, region, or volume) 111. In some embodiments, the second chamber 111 can be referred to as a “test microorganisms growth chamber” or a “detection chamber,” and can include a volume to be interrogated for test microorganisms viability to determine the efficacy of a sterilization process.

The first chamber 109 and the second chamber 111 can be positioned in fluid communication with each other to allow a sterilant and the liquid 122 to move from (i.e., through) the first chamber 109 to the second chamber 111. In some embodiments, the degree of fluid connection between the first chamber 109 and the second chamber 111 (e.g., the size of an opening, such as the opening 117, connecting the first chamber 109 and the second chamber 111) can increase after, simultaneously with, and/or in response to the activation step (i.e., the liquid 122 being released from the container 120). In some embodiments, the control of fluid communication (or extent of fluid connection) between the first chamber 109 (e.g., in the upper portion 116) and the second chamber 111 (e.g., in the lower portion 114) can be provided by at least a portion of the insert 130.

The container 120 can be positioned and held in the first chamber 109 during sterilization and when the container 120 is in a first, unfractured, state. The test microorganisms 115 can be housed in the second chamber 111 and in fluid communication with ambience when the container 120 is in the first state. The first chamber 109 and the second chamber 111 can be configured such that the container 120 is not present in the second chamber 111, and particularly, not when the container 120 is in its first, unfractured, state. A sterilant can move into the second chamber 111 (e.g., via the first chamber 109) during sterilization, and the liquid 122 can move into the second chamber 111 (e.g., from the first chamber 109) during activation, when the container 120 is fractured and the liquid 122 is released into the interior of the housing 102.

As a result, when the container 120 is in the first state, the first chamber 109 and the second chamber 111 can be in fluid communication with one another, and with ambience (e.g., during sterilization). For example, the first chamber 109 and the second chamber 111 can be in fluid communication with ambience via the one or more apertures 107. In some embodiments, the first chamber 109 and the second chamber 111 can be in fluid communication with ambience in such a way that the first chamber 109 is positioned upstream of the second chamber 111 when a sterilant is entering the biological indicator 100. That is, the first chamber 109 can be positioned between the sterilant inlet (e.g., the one or more apertures 107) and the second chamber 111, and the sterilant inlet can be positioned on an opposite side of the first chamber 109 than the second chamber 111.

### Systems

In another aspect, the present disclosure provides a system that can be used for determining the efficacy of a sterilization process. The system comprises any embodiment of the self-contained biological indicator according to the present invention, and an automated reader. The automated reader is configured to i) receive at least a portion of the biological indicator, ii) direct a first wavelength of electromagnetic radiation into the liquid composition in the housing, and iii) detect or measure a quantity of a second wavelength of electromagnetic radiation emitted by the :fluorescent product. Accordingly, a person having ordinary skill in the art will recognize the automated reader comprises inter alia a locus (e.g., a chamber) dimensioned to receive the biological indicator, a source of ultraviolet electromagnetic radiation, a photodetector for detecting and measuring fluorescence emitted from the biological indicator, at least one microprocessor for controlling components of the automated reader. Optionally, the automated reader further comprises software or firmware comprising an algorithm for identifying biological indicators that exhibit fluorescence indicative of complete inactivation the test microorganisms or survival of at least a portion of the test microorganisms after exposure to a sterilization process.

In any embodiment of the system, the self-contained biological indicator is adapted, as disclosed herein, to be used to determine efficacy of any steam sterilization process selected from the group consisting of 121° C. gravity process, 121° C. pre-vac process, 121° C. SFPP process, 132° C. gravity process, 132° C. pre-vac process, 132° C. SFPP process, 134° C. pre-vac process, 134° C. SFPP process, 135° C. gravity process, 135° C. pre-vac process, and 135° C. SFPP process.

### Housing

In general, the housing refers to a container, usually an outer container, having walls impermeable to a sterilant, where other components of the biological indicator are located. The housing may be inside a process challenge device or may be a process challenge device itself. In some embodiments, the housing may have dimensions useful to produce a flat or generally planar biological indicator. This disclosure encompasses housings of any shape and dimensions.

The housing contains at least one opening that allows flow of a sterilant to the interior of the housing (sterilant pathway). In some embodiments, the housing may comprise a body with an opening and a cap to close that opening. In some embodiments, the cap may be capable of completely sealing the housing and eliminating any fluid communication between the interior of the housing and ambiance (e.g., closing the sterilant pathway). In general, the cap has an open position in which there is an opening (e.g., a gap) between the cap and the body of the container that allows flow of liquid or gas (e.g., a sterilant) into and out of the interior of the housing. The cap also has a closed position where the opening is sealed and any fluid flow through the gap is eliminated. In other embodiments, the cap may comprise vents that allow passage of a sterilant to the interior of the housing and create an additional sterilant pathway, even if the cap is present and in the closed position. In other preferred embodiments, however, when the cap comprises vents, placing the cap in the closed position simultaneously closes: (a) the gap between the cap and the body of the container and (b) the vents present on the cap, essentially closing the sterilant pathway.

In other embodiments, the cap may lack vents and the only sterilant pathway may be through the space between the cap and the body of the housing (or through another opening or vent, if present on the body) when the cap is the open position. In some embodiments, if vents exist on the housing, they are located on the cap. In embodiments where no other opening exists besides the opening between the cap and the body of the housing, then placing the cap in the closed position completely seals off the interior of the housing, which stops the fluid communication between the interior of the housing and ambience. In those embodiments, the sterilant pathway may be sealed when the cap is in the closed position.

### Test Microorganisms

Articles of the present disclosure comprise a test microorganism. In certain embodiments, the test microorganism may be a plurality of test microorganisms. In certain embodiments, the test microorganism may be a plurality of spores. Suitable test microorganisms used in self-contained biological indicators are well known in the art. The test microorganisms comprise and/or are capable of producing an enzyme capable of catalyzing a cleavage of an enzyme substrate, which can be used to detect test microorganisms that have survived exposure to a sterilization process.

Preferred microorganisms comprising an enzyme useful in the practice of the present invention are bacteria or fungi in either the spore or vegetative state. Particularly preferred test microorganisms include, without limitation, Bacillus, Clostridium, Neurospora, and Candida species of microorganisms.

Methods of the present invention may include the step of incubating any of the microorganisms which remain viable, following the completion of the sterilization cycle, with an aqueous nutrient medium. Inclusion of this step confirms by conventional techniques whether the sterilization conditions had been sufficient to kill all of the microorganisms in the indicator, indicating that the sterilization conditions had been sufficient to sterilize all of the items in the sterilizer. If growth of the microorganism is used in a conventional manner to confirm the results of a rapid enzyme test, the microorganism should be one which is conventionally used to monitor sterilization conditions. These conventionally used microorganisms are generally many times more resistant to the sterilization process being employed than most organisms encountered in natural contamination. The bacterial spore is recognized as the most resistant form of microbial life. It is the life fom1 of choice in all tests for determining the sterilizing efficacy of devices, chemicals and processes. Spores from Bacillus and Clostridium species are the most commonly used to monitor sterilization processes utilizing saturated steam, dry heat, gamma irradiation and ethylene oxide.

Generally, test microorganisms chosen to be used in a biological indicator are particularly resistant to a given sterilization process. In certain embodiments, the biological indicators of the present disclosure include a viable culture of a known species of microorganism, usually in the form of microbial spores. Spores (e.g., bacterial spores), rather than the vegetative form of the microorganisms, are used at least partly because vegetative microorganisms are known to be relatively easily killed by sterilizing processes. Additionally, spores also have superior storage characteristics and could remain in their dormant state for years. As a result, sterilization of an inoculum of a standardized spore strain provides a higher degree of confidence that inactivation of all microorganisms in a sterilizing chamber has occurred.

By way of example only, the present disclosure describes the microorganisms used in the biological indicator as being “spores;” however, it should be understood that the type of microorganism (e.g., spore) used in a particular embodiment of the biological indicator is selected for being resistant to the particular sterilization process contemplated (more resistant than the microorganisms normally present on the items to be sterilized so that inactivation of the test microorganisms indicates a successful sterilization.). Accordingly, different embodiments of the present disclosure using different sterilants may use different microorganisms, depending on the sterilization process for which the particular embodiment is intended.

In general, the spores used in a particular system are selected according to the sterilization process at hand. For example, for a steam sterilization process, Geobacillus stearothermophilus or Bacillus stearothermophilus can be used. In another example, for an ethylene oxide sterilization process, Bacillus atrophaeus (formerly Bacillus subtilis) can be used. In some embodiments, the spores can include, but are not limited to, at least one of Geobacillus stearothermophilus, Bacillus stearothermophilus, Bacillus subtilis, Bacillus atrophaeus, Bacillus megaterium, Bacillus coagulans, Clostridium sporogenes, Bacillus pumilus, or combinations thereof.

### Enzymes and Enzyme Substrates

The test microorganisms either comprise an enzyme capable of catalyzing the cleavage of an enzyme substrate to produce a fluorescently detectable compound, or are capable of producing such an enzyme, or both. The enzymes useful in biological indicators of the present disclosure include extracellular and intracellular enzymes whose activity correlates with the viability of at least one of the microorganisms commonly used to monitor sterilization efficacy (“test” microorganism or “test spores”). In this context, “correlates” means that the enzyme activity, over background, can be used to predict growth of the test microorganism. The enzyme should be one which, following a sterilization cycle which is sublethal to the test microorganism, remains sufficiently active to react with a substrate for the enzyme, within twenty-four hours, and in preferred embodiment within eight hours or less, yet be inactivated or substantially reduced in activity following a sterilization cycle which would be lethal to the test microorganism.

Detection of the enzyme activity, after the test microorganisms has been exposed to a sterilization process, affords a more rapid detection of surviving test microorganisms than traditional growth-based detection methods.

Examples of suitable enzymes include α-glucosidase, α-galactosidase, lipase, esterase, acid phosphatase, alkaline phosphatase, proteases, aminopeptidase, chymotrypsin, β-glucosidase, β-galactosidase, α-glucoronidase, β-glucoronidase, phosphohydrolase, plasmin, thrombin, trypsin, calpain, α-mannosidase, β-mannosidase, α-L-fucosidase, leucine aminopeptidase, α-L-arabinofuranosidase, cysteine aminopeptidase, valine aminopeptidase, β-xylosidase, α-L-iduronidase, glucanase, cellobiosidase, cellulase, α-arabinosidase, glycanase, sulfatase, butyrase, glycosidase, arabinoside, and a combination of any two or more of the foregoing enzymes. In certain embodiments of the articles, kits, systems and methods of the present disclosure, the source of biological activity used therein comprises an isolated or otherwise purified form of any of the foregoing suitable enzymes.

In the context of this application, an enzyme substrate comprises a substance or mixture of substances that, when acted upon by an enzyme, are converted into an enzyme-modified product. Although the preferred substrate produces a fluorescently detectable compound, in other embodiments, the product of the enzymatic action may be a luminescent or colored material. In other embodiments, however, the enzyme substrate can consist of a compound which when reacted with the enzyme, will yield a product that will react with an additional compound or composition to yield a luminescent, fluorescent, or colored material. Preferably, if the substrate is to be included in the indicator device during sterilization, the substrate should not spontaneously break down or convert to a detectable product during sterilization or incubation. For example, in devices used to monitor steam and dry heat sterilization, the substrate must be stable at temperatures between about 20° C. and 180° C. Preferably also, where the enzyme substrate is to be included with conventional growth media, it must be stable in the growth media, e.g., not autofluoresce in the growth media.

In general, there are two basic types of enzyme substrates that can be used in the biological indicators of this disclosure. The first type of substrate can be either fluorogenic (or chromogenic), and can be given a chemical formula such as, AB. When acted upon by the enzyme, AB breaks down into the products A and B. B, for example, could be either fluorescent or colored. A specific example of a fluorogenic substrate of this type are derivatives of 4-methylumbelliferone. Other fluorogenic substrates of this type include the derivatives of 7-amido-4-methylcoumarin (7-AMC), indole and fluorescein. An example of a chromogenic substrate of this type is 5-bromo-4-chloro-3-indolyl phosphate. In the presence of phosphatase, the substrate will be broken down into indigo blue and phosphate. Other chromogenic substrates of this type include derivatives of 5-bromo-4-chloro-3-indolyl, nitrophenol and phenolphthalein, listed below.

The second type of substrate can be given the chemical formula CD, for example, which will be converted by a specific enzyme into C and D. In this case, however, neither C nor D will be fluorescent or colored, but either C or D is capable of being further reacted with compound Z to give a fluorescent or colored compound, thus indicating enzyme activity. A specific fluorogenic example of this type is the amino acid lysine. In the presence of the enzyme lysine decarboxylase, lysine loses a molecule of CO₂. The remaining part of the lysine is then called cadaverine, which is strongly basic. A basic indicator such as 4-methylumbelliferone can be incorporated and will fluoresce in the presence of a strong base. A chromogenic substrate of this type would be 2-naphthyl phosphate. The enzyme phosphatase reacts with the substrate to yield β-naphthol. The liberated β-naphthol reacts with a chromogenic reagent containing 1-diazo-4-benzoylamino-2,5-diethoxybenzene, commercially available as “Fast Blue BB Salt” from Sigma Chemical, to produce a violet color.

As mentioned above, a preferred enzyme substrate in some embodiments is a fluorogenic substrate, defined herein as a compound capable of being enzymatically modified, e.g., by hydrolysis or other enzymatic action, to give a derivative fluorophore that has a measurably modified or increased fluorescence.

A person having ordinary skill in the art would understand that suitable fluorogenic compounds are in themselves either non-fluorescent or meta-fluorescent (i.e., fluorescent in a distinctly different way e.g., either by color or intensity, compared to the corresponding enzyme-modified products). In that regard, appropriate wavelengths of excitation and detection, in a manner known to users of fluorometric techniques, are used to separate the fluorescence signal developed by the enzyme modification from any other fluorescence that may be present.

Non-limiting examples of suitable enzymatic substrates can include, for example, derivatives of coumarin including 7-hydroxycoumarin (also known as umbelliferone or 7-hydroxy-2H-chromen-2-one) derivatives and 4-methylumbelliferone (7-hydroxy-4-methylcoumarin) derivatives including:4-methylumbelliferyl-α-D-glucopyranoside, 4-methylumbelliferyl-α-D-galactopyranoside, 4-methylumbelliferyl heptanoate, 4-methylumbelliferyl palmitate, 4-methylumbelliferyl oleate, 4-methylumbelliferyl acetate, 4-methylumbelliferyl nonanoate, 4-methylumbelliferyl caprylate, 4-methylumbelliferyl butyrate, 4-methylumbelliferyl-β-D-cellobioside, 4-methylumbelliferyl acetate, 4-methylumbelliferyl phosphate, 4-methylumbelliferyl sulfate, 4-methylumbelliferyl-β-trimethylammonium cinnamate chloride, 4-methylumbelliferyl-β-D-N,N′,N″-triacetylchitotriose, 4-methylumbelliferyl-β-D-xyloside, 4-methylumbelliferyl-N-acety-1-β-D-glucosaminide, 4-methylumbelliferyl-N-acetyl-α-D-glucosaminide, 4-methylumbelliferyl propionate, 4-methylumbelliferyl stearate, 4-methylumbelliferyl-α-L-arabinofuranoside, 4-methylumbelliferyl-α-L-arabinoside; 4-methylumbelliferyl-β-D-N,N′-diacetyl chitobioside, 4-methylumbelliferyl elaidate, 4-methylumbelliferyl-α-D-mannopyranoside, 4-methylumbelliferyl-β-D-mannopyranoside, 4-methylumbelliferyl-β-D-fucoside, 4-methylumbelliferyl-α-L-fucoside, 4-methylumbelliferyl-β-L-fucoside, 4-methylumbelliferyl-α-D-galactoside, 4-methylumbelliferyl-β-D-galactoside, 4-trifluoromethylumbelliferyl-β-D-galactoside, 4-methylumbelliferyl-α-D-glucoside, 4-methylumbelliferyl-β-D-glucoside, 4-methylumbelliferyl-7,6-sulfo-2-acetamido-2-deoxy-β-D-glucoside, 4-methylumbelliferyl-β-D-glucuronide, 6,8-difluor-4-methylumbelliferyl-β-D-glucuronide, 6,8-difluoro-4-methylumbelliferyl-β-D-galactoside, 6,8-difluoro-4-methylumbelliferyl phosphate, 6,8-difluoro-4-methylumbelliferyl-β-D-xylobioside, for example. The second substrate can also be derivatives of 7-amido-4-methylcoumarin, including: Ala-Ala-Phe-7-amido-4-methylcoumarin, Boc-Gln-Ala-Arg-7-amido-4-methylcoumarin hydrochloride, Boc-Leu-Ser-Thr-Arg-7-amido-4-methylcoumarin, Boc-Val-Pro-Arg-7-amido-4-methylcoumarin hydrochloride, D-Ala-Leu-Lys-7-amido-4-methylcoumarin, L-alanine 7-amido-4-methylcoumarin trifluoroacetate salt, L-methionine 7-amido-4-methylcoumarin trifluoroacetate salt, L-tyrosine 7-amido-4-methylcoumarin, Lys-Ala-7-amido-4-methylcoumarin dihydrochloride, N-β-Tosyl-Gly-Pro-Arg 7-amido-4-methylcoumarin hydrochloride, N-succinyl-Ala-Ala-Phe-7-amido-4-methylcoumarin, N-succinyl-Ala-Ala-Pro-Phe-7-amido-4-methylcoumarin, N-succinyl-Ala-Phe-Lys 7-amido-4-methylcoumarin acetate salt, N-succinyl-Leu-Leu-Val-Tyr-7-Amido-4-methylcoumarin, D-Val-Leu-Lys 7-amido-4-methylcoumarin, Fmoc-L-glutamic acid 1-(7-amido-4-methylcoumarin), Gly-Pro-7-amido-4-methylcoumarin hydro bromide, L-leucine-7-amido-4-methylcoumarin hydrochloride, L-proline-7-amido-4-methylcoumarin hydrobromide; other 7-hydroxycoumarin derivatives including 3-cyano-7-hydroxycoumarin (3-cyanoumbelliferone), and 7-hydroxycoumarin-3-carboxylic acid esters such as ethyl-7-hydroxycoumarin-3-carboxylate, methyl-7-hydroxycoumarin-3-carboxylate, 3-cyano-4-methylumbelliferone, 3-(4-imidazolyl)umbelliferone; derivatives of fluorescein including: 2′,7′-bis-(2-carboxyethyl)-5-(and -6-)carboxyfluorescein, 2′,7′-bis-(2-carboxypropyl)-5-(and-6-)-carboxyfluorescein, 5- (and 6)-carboxynaphthofluorescein, anthofluorescein, 2′,7′-dichlorofluorescein diacetate, 5(6)-carboxyfluorescein, 5(6)-carboxyfluorescein diacetate, 5-(bromomethyl)fluorescein, 5-(iodoacetamido)fluorescein, 5-([4,6-dichlorotriazin-2-yl]amino)fluorescein hydrochloride, 6-carboxyfluorescein, eosin Y, fluorescein diacetate 5-maleimide, fluorescein-O′-acetic acid, O′-(carboxymethyl)fluoresceinamide, anthofluorescein, rhodols, halogenated fluorescein; derivatives of rhodamine including: tetramethylrhodamine, carboxy tetramethyl-rhodamine, carboxy-X-rhodamine, sulforhodamine 101 and rhodamine B; afluorescamine derivatives; derivatives of benzoxanthene dyes including: seminaphthofluorones, carboxy-seminaphthofluorones seminaphthofluoresceins, seminaphthorhodafluors; derivatives of cyanine including sulfonated pentamethine and septamethine cyanine.

In some embodiments, the enzyme whose activity is to be detected may be chosen from α-D-glucosidase, chymotrypsin, or fatty acid esterase. In the case of Bacillus stearothermophilus, the fluorogenic enzyme substrate is preferably 4-methylumbelliferyl-α-D-glucoside, 7-glutarylphenylalanine-7-amido-4-methyl coumarin, or 4-methylumbelliferyl heptanoate. If the enzyme whose activity is to be detected is α-L-arabinofuranosidase, e.g., derived from Bacillus atrophaeus, a preferred fluorogenic enzyme substrate is 4-methylumbelliferyl-α-L-arabinofuranoside. In preferred embodiments, 4-methylumbelliferyl α-D-glucopyranoside is the enzyme substrate used to produce the metabolic activity and the enzyme is a glucosidase, such as f3-D-glucosidase.

### Salt Compound

A self-contained biological indicator of the present disclosure comprises an effective amount of a salt compound disposed in the housing. Suitable salt compounds can include the salt of any ion selected from the group consisting of acetate; borate; citrate; carbonate; bicarbonate; phosphate; hydrogen phosphate; dihydrogen phosphate; chloride; sulfate; N,N-bis(2-hydroxyethyl)-2-aminoethanesulfonate; N,N-bis(2-hydroxyethyl)glycine; 3-(cyclohexylamino)-2-hydroxy-1-propanesulfonic acid; N-cyclohexyl-2-aminoethanesulfonate; imidazolium; 2-(N-morpholino)ethanesulfonate; 3-(N-morpholino)propanesulfonic acid; tricine, 2-amino-2-(hydroxymethyl)propane-1,3-diol; and a combination of any two or more of the foregoing salts.

The salt compound comprises one or more compounds that do not substantially inhibit the growth, germination, or detection of spores and/or test microorganisms in the resulting mixture. Alternatively, or additionally, the salt compound comprises one or more salt compounds that do not substantially inhibit the detection of the detectable enzyme activity in the resulting mixture.

The salt compound may be disposed in the housing in the liquid composition. Alternatively, or additionally, the salt compound may be present in the housing mixed, and optionally dried, with the test microorganism. In some embodiments, the salt compound may comprise a buffering agent. In some embodiments, the salt compound can be disposed in the liquid composition. In some embodiments, the salt compound can be disposed in the housing separated from the liquid composition. In some embodiments, the salt compound can be disposed in a dry form separated from or mixed with the test microorganisms. In some embodiments, the salt compound can be disposed in the liquid composition and be disposed in the housing separated from the liquid composition.

The effective amount of the salt compound is determined by the final concentration of the salt compound when it is mixed with the liquid composition of the self-contained biological indicator. Suitable concentrations of the salt compound in the liquid composition are disclosed hereinbelow.

### Liquid Composition

The liquid composition is located in the frangible container and contains one or more of the enzyme substrates mentioned above. In certain embodiments, the enzyme substrate is 4-methylumbelliferyl-α-D-glucoside (MUG). In some embodiments, the liquid composition may also include nutrients for the test microorganisms (e.g., spores), such as germination nutrients that allow germination and/or growth of any viable surviving spores. In some preferred embodiments, the solvent of the liquid composition is water. A combination of nutrients form a nutrient medium and together with the enzyme substrate and other non-nutrient components (such as a pH indicators) form the liquid composition.

Suitable nutrients may be provided initially in a dry form (e.g., powdered form, tablet form, caplet form, capsule form, a film or coating, entrapped in a bead or other carrier, another suitable shape or configuration, or a combination thereof) and then combined with a suitable solvent to provide a liquid composition that is then placed in the frangible container.

The nutrients in the liquid medium can include one or more sugars, including, but not limited to, glucose, fructose, dextrose, maltose, trehalose, cellobiose, or the like, or a combination thereof. Alternatively, the nutrients may include complex media, such as peptone, tryptone, phytone peptone, yeast extract, soybean casein digest, other extracts, hydrolysates, etc., or a combination thereof. In other embodiments, the nutrients in the liquid composition represent a combination of one or more complex media components and other specific nutrients. The nutrient medium may also include the salt compound of the present disclosure. In some embodiments, the nutrient can further include at least one amino acid, including, but not limited to, at least one of methionine, phenylalanine, alanine, tyrosine, and tryptophan.

As part of a self-contained biological indicator, the liquid composition optionally comprising nutrients, the enzyme substrate, and/or other components is typically present throughout the sterilization procedure but is kept separate and not accessible to the test microorganisms in the frangible container until desired. After the sterilization process is completed and the biological indicator is used to determine the efficacy of the sterilization, the liquid composition is placed in contact with the test microorganisms resulting in a mixture. In this disclosure, placing the liquid composition in contact with the spores includes activating (e.g., fracturing or otherwise opening) the frangible container so that the liquid composition is released and contacts the test microorganisms. This process may include mixing the liquid composition with the test microorganisms, such as manual or mechanical shaking of the housing of the biological indicator so that the liquid composition adequately mixes with the spores.

In this disclosure, the process of bringing the test microorganisms (e.g., spores) and medium together is referred to as “activation” of the biological indicator. That is, the term “activation” and variations thereof, when used with respect to a biological indicator refer generally to bringing one or more test microorganisms (e.g., spores) in fluid communication with the liquid composition (comprising, e.g., a nutrient medium for the spores of interest and an enzyme substrate). For example, when a frangible container within the biological indicator that contains the liquid composition is at least partially fractured, punctured, pierced, crushed, cracked, breaking, or the like, such that the medium has been put in fluid communication with the test microorganisms, the biological indicator can be described as having been “activated.” Said another way, a biological indicator has been activated when the test microorganisms have been exposed to the liquid composition that was previously housed separately from the test microorganisms.

In some embodiments, the mixture resulting from mixing the liquid composition with the test microorganisms after activation remains isolated within the housing of the biological indicator after the sterilization cycle has been completed and no additional reagents or components are added to it during or after activation. If the test microorganisms are viable and grow, then the enzyme produced by the microorganisms catalyzes the cleavage of the enzyme substrate, which produces the fluorescently-detectable compound. This means that the same solution in the same container (housing) is used for three separate events: (a) test microorganism germination and/or growth, if the test microorganisms are viable, (b) the enzymatic cleavage of the enzyme substrate, resulting in the production of the fluorescently-detectable compound, and (c) the fluorescence detection of the fluorescently-detectable compound.

The inventors have developed a liquid composition for the self-contained biological indicator such that the three events mentioned above can take place in the same container, using the same germination/growth solution for the cleavage and the florescence detection. In certain embodiments, the liquid composition comprises the test microorganism(s), a fluorogenic enzyme substrate, a nutrient that facilitates germination and or growth of the test microorganisms, and an additional salt compound that facilitates detection of the fluorescently-detectable compound produced by a reaction involving the fluorogenic enzyme substrate and an enzyme associated with the test microorganism.

It was shown previously (U.S. Provisional Patent Application No. 62/711,007 (Attorney Docket No. 79760US002) filed on Jul. 27, 2018) that adjusting the pH of a liquid composition used in a self-contained biological indicator can facilitate detection of a fluorescently-detectable compound used in the biological indicator. It is now known that the addition of a salt compound can have an additive effect (beyond that of the pH) and thereby improve the detection of the enzyme that catalyzes the enzyme substrate to produce the fluorescently detectable compound (e.g., by increasing the activity of the enzyme and/or by stabilizing the fluorescent signal and/or by causing an improved correlation of the detection of the enzyme activity and the detection of growth of the test microorganisms after the biological indicator is exposed to a sterilization process).

In certain embodiments of the articles, systems, or methods of the present disclosure, suitable enzyme substrates include, but are not limited to, enzyme substrates that comprise a fluorophore component selected from the group consisting of 4-methyl-5-fluoro-2H-chromen-2-one, 4-methyl-6-fluoro-2H-chromen-2-one, 4-methyl-8-fluoro-2H-chromen-2-one, 4-methyl-6,8-difluoro-2H-chromen-2-one, 4-methyl-6-chloro-2H-chromen-2-one, and 4-methylethanoate-6-fluoro-2H-chromen-2-one.

In some embodiments, the liquid composition may comprise the salt compound. Alternatively or additionally, the salt compound can be provided in the housing of the biological indicator as a dry powder, optionally mixed with the test microorganisms. In these alternative embodiments, the salt compound readily combines with the liquid composition when the biological indicator is activated as described herein.

The ionic conditions (e.g., concentration) of the salt compound, when dissolved in the liquid composition, should be such that the enzyme and enzyme substrate are not substantially affected in a way that hinders detection of the enzyme activity. In some embodiments, the salt compound is used as part of the liquid composition, such as phosphate buffers, (e.g., phosphate buffered saline solution, potassium phosphate or potassium phosphate dibasic), tris(hydroxymethyl) aminomethane-HCl solution, or acetate buffer, or any other buffer suitable for sterilization known in the art. Salt compounds suitable for the present biological indicators should be compatible with fluorogenic and chromogenic enzyme substrates used as part of the liquid composition. Another consideration in choosing the salt compound is their influence on the enzyme activity. For example, a phosphate buffer may contain a relatively high concentration of inorganic phosphate, which is a competitive inhibitor of alkaline phosphatase. Thus, for that enzyme, a Tris-HCl buffer is recommended.

In certain embodiments, the concentration of the salt compound, when dissolved in the liquid composition (before or after activation of the biological indicator), may be at least about 0.5 mM, at least about 1.0 mM, at least about 2.0 mM, at least about 3.0 mM, at least about 4.0 mM, at least about 5.0 mM, at least about 7.5 mM, at least about 10.0 mM, at least about 15 mM, at least about 20 mM, at least about 25 mM, at least about 30 mM, at least about 40 mM, or at least about 45 mM. In certain embodiments, the concentration of the salt compound, when dissolved in the liquid composition (before or after activation of the biological indicator), may be greater than 0.5 mM, greater than 1.0 mM, greater than 2.0 mM, greater than 3.0 mM, greater than 4.0 mM, greater than 5.0 mM, greater than 7.5 mM, greater than 10.0 mM, greater than 15 mM, greater than 20 mM, greater than 25 mM, greater than 30 mM, greater than 40 mM, or greater than 45 mM. In certain embodiments, the concentration of the salt compound, when dissolved in the liquid composition (before or after activation of the biological indicator), may be up to about 5 mM, up to about 10 mM, up to about 15 mM, up to about 20 mM, up to about 25 mM, up to about 30 mM, up to about 35 mM, up to about 40 mM, up to about 45 mM, or up to about 50 mM. In certain embodiments, the concentration of the salt compound, when dissolved in the liquid composition (before or after activation of the biological indicator), may be less than 5 mM, less than 10 mM, less than 15 mM, less than 20 mM, less than 25 mM, less than 30 mM, less than 35 mM, less than 40 mM, less than 45 mM, or less than 50 mM. In certain embodiments, the concentration of the salt compound, when dissolved in the liquid composition (before or after activation of the biological indicator), may be from about 0.5 mM to about 50 mM, from about 0.5 mM to less than 10 mM, or from greater than 10 mM to about 50 mM. In any embodiment, when the concentration of the salt compound, dissolved in the liquid composition (before or after activation of the biological indicator) is 10 mM, the salt compound is not potassium phosphate.

The concentration of enzyme substrate present in the liquid composition depends upon the identity of the particular substrate and enzyme, the amount of enzyme-product that must be generated to be detectable, either visually or by instrument, and the amount of time that one is willing to wait in order to determine whether active enzyme is present in the reaction mixture. Preferably, the amount of enzyme substrate is sufficient to react with any residual active enzyme present, after the sterilization cycle, within about an eight-hour period of time, such that at least 10- molar enzyme-modified product is produced. In cases where the enzyme substrate is a 4-methylumbelliferyl derivative, the inventors have been found that its concentration in the aqueous buffered solution is preferably between about 10-5 and 10-3 molar.

Although the use of a buffered solution may aid in providing stable reaction conditions for the enzyme and its substrate, a buffered solution is not required. Accordingly, in some embodiments, the liquid composition only comprises a solution adjusted to a suitable pH, but without an added buffer system. In other embodiments, however, the liquid composition does comprise a buffered solution.

In some embodiments, the biological indicator may comprise an additional indicator compound that can facilitate the detection of another metabolic activity of the test microorganisms (e.g., spore) (aside from an enzyme substrate that can produce a fluorescently-detectable compound).

This additional metabolic activity can also be an enzymatic activity. Non-limiting examples of indicator compounds include a chromogenic enzyme substrate (e.g., observable in the visible spectrum), a pH indicator, a redox indicator, a chemiluminescent enzyme substrate, a dye, and a combination of any two or more of the foregoing indicator compounds.

In some embodiments, the additional indicator is a pH indicator that produces a change in color when the pH decreases, indicating growth of the test microorganisms. In some embodiments, the pH indicator is bromocresol purple. The pH indicator can be used to detect a second biological activity, such as the fermentation of a carbohydrate to acid end products (suggesting survival of the test microorganisms) and an enzymatic biological activity such as α-D-glucosidase enzyme activity, for example. These activities can indicate the presence or absence of a viable test microorganism following the exposure of a biological indicator to a sterilization process, for example. The bromocresol purple can be used at a concentration of about 0.03 g/L in the aqueous mixture, for example. The 4-methylumbelliferyl-α-D-glucoside can be used, for example, at a concentration of about 0.05 to about 0.5 g/L (e.g., about 0.05 g/L, about 0.06 g/L, about 0.07 g/L, about 0.08 g/L, about 0.09 g/L, about 0.1 g/L, about 0.15 g/L, about 0.2 g/L, about 0.25 g/L, about 0.3 g/L, about 0.35 g/L, about 0.4 g/L, about 0.45 g/L, about 0.5 g/L) in the aqueous mixture.

The combination of bromocresol purple and 4-methylumbelliferyl-α-D-glucoside represents a preferred combination of enzymatic substrate and additional indicator according to the present disclosure, but other combinations are contemplated within the scope of the present disclosure. In yet other embodiments, the biological indicator does not comprise a pH indicator.

In some situations, one or more components of a biological indicator (e.g., crevasses in the housing, substrates or carriers for spores, walls of container, etc.) may retain residual oxidizing sterilant. This can occur, for example, with hydrogen peroxide vapor as well as with other vapor sterilants such as ozone and peracetic acid. For example, certain carrier materials, e.g., those that are hydrophilic such as glass fiber and cellulosic materials, can retain residual oxidizing sterilant, particularly hydrogen peroxide. In this context, “residual” means an amount of retained sterilant that inhibits the growth of low numbers of spore survivors. Typically, this means more than 10 micrograms of sterilant retained per microgram of carrier. In certain situations, the amount of residual sterilant can be greater than 40 micrograms sterilant per milliliter of growth media. As a comparison, if the carrier material has a contact angle of greater than 90°, it is hydrophobic, and there is generally no more than 10 micrograms sterilant retained per microgram of carrier.

Therefore, in some embodiments, the biological indicators comprise one or more neutralizers, which are not an enzyme and not a metal catalyst disposed within the biological indicator. A neutralizer is a compound or material that reacts with residual sterilant, e.g., hydrogen peroxide, to neutralize its effect, wherein the neutralizer is not an enzyme, and not a metal catalyst. Enzyme neutralizers are typically not stable at the high temperatures, and thus not desirable.

Suitable examples of neutralizers include sulfur containing materials such as methionine, L-cysteine, D-ethionine, S-methyl-L-cysteine, S-benzyl-L-cysteine, sodium thiosulfate, glutathionine, L-cystathionine, N-acetyl-L-cysteine, carboxymethylcysteine, D,L-homocysteine, D,L-homocysteine-thiolactone, and thiodipropionic acid, and non-sulfur containing materials such as isoascorbic acid, potassium ferricyanide, and sodium pyruvate. Various combinations of such neutralizers can be used. Preferred neutralizers include methionine, L-cysteine, D-ethionine, S-methyl-L-cysteine, S-benzyl-L-cysteine, sodium thiosulfate, thiodipropionic acid, isoascorbic acid, potassium ferricyanide, sodium pyruvate, and combinations thereof.

Sterilization Processes Biological indicators of the present disclosure may be used to monitor the effectiveness of one or more types of sterilization procedures, including sterilization procedures that use various sterilants, such as steam (e.g., pressurized steam), vapor phase hydrogen peroxide (which may or may not include hydrogen peroxide plasma), ethylene oxide gas, dry heat, propylene oxide gas, methyl bromide, chlorine dioxide, formaldehyde and peracetic acid (alone or with a vapor phase of another material), ozone, radiation, and combinations thereof.

In at least some of the sterilization processes, an elevated temperature, for example, 50° C., 60° C., 100° C., 121° C., 132° C., 134° C., 135° C. or the like, is included or may be encountered in the process. In addition, elevated pressures and/or a vacuum may be encountered, for example, 15 psi (1×10⁵ Pa) at different stages within a single given sterilization cycle, or in different sterilization cycles.

In the case of steam being the sterilant, the sterilization temperatures can include 121° C., 132° C., 134° C., 135° C. The instant biological indicators are suitable for steam sterilization cycles at each of the temperatures above and for each temperature the cycle can have a different air removal process chosen from gravity, prevacuum (“pre-vac”), and steam flush pressure pulse (SFPP). Each of these cycles may have different exposure times depending on the type of instruments/devices being sterilized. In this disclosure, prevacuum and SFPP are also labeled as Dynamic Air Removal (DAR) cycles.

A tabular representation of exemplary steam sterilization cycles in which the present biological indicators can be used is shown below:

| 121° C. | | | 132° C. | | | 134° C. | | | 135° C. | | |
|---|---|---|---|---|---|---|---|---|---|---|---|
| Gravity | Pre-Vac | SFPP | Gravity | Pre-Vac | SFPP | Gravity | Pre-Vac | SFPP | Gravity | Pre-Vac | SFPP |

In this disclosure, the term a “T gravity” sterilization cycle refers to a steam process where the sterilization temperature is TC and where air is removed (conditioning) from the sterilization chamber as a result of steam displacement. In this case, the force of gravity causes the heavier gas (air) to exit the chamber via the sterilizer drain as steam enters the chamber. In general, gravity cycles require more exposure time because the air removal method is more passive in nature. For instance, a “121 gravity” cycle is a steam sterilization carried out at 121° C. under gravity conditioning.

A “T pre-vac” sterilization cycle refers to a steam process where the sterilization temperature is T° C. and where air removal is done by mechanical vacuum evacuation in conjunction with steam injections. As a consequence of this conditioning method, the pressure in the sterilization chamber can decrease below atmospheric values during the evacuation cycle and can increase to positive pressures when steam is being introduced. For instance, “121 pre-vac” sterilization cycle refers to a steam process where the sterilization temperature is 121° C. and the conditioning occurs via vacuum evacuations.

A “T SFPP” sterilization cycle refers to a steam process where the sterilization temperature is T° C. and where conditioning is carried out through a series of pressurizations and flushes with steam. During a SFPP process, the pressure in the chamber does not drop below atmospheric (no vacuum is drawn). For example, a “121 SFPP cycle refers to a steam process where the sterilization temperature is 121° C. and the conditioning occurs via steam flush pressure pulses.

In this disclosure, a “dynamic air removal” cycle refers to a sterilization cycle that uses either pre-vacuum or SFPP conditioning.

In other embodiments, the biological indicators of the present disclosure may be used to monitor the effectiveness of a vapor phase sterilization procedure that uses an oxidizing sterilant. In some embodiments, the biological indicators may be used to monitor the effectiveness of any of the hydrogen peroxide sterilization procedures known in the art. More preferably, the biological indicator may be used to monitor the effectiveness of a hydrogen peroxide vapor phase sterilization procedure.

While aqueous hydrogen peroxide (H₂O₂) has a long history of use as a sterilant, the concept of vapor-phase hydrogen peroxide (VPHP) sterilization has been developed within the past decade. This process is a low temperature sterilization process that kills a wide range of microorganisms including bacterial endospore-forming bacteria commonly used as challenge organisms to evaluate and validate the effectiveness of sterilization cycles in hospitals. A major advantage of hydrogen peroxide is its short exposure cycle time (few minutes). Furthermore, at the end of a hydrogen peroxide sterilization process, only air and water remain in the chamber. Significantly, the novel features of the biological indicators described herein allow for the development of a rapid-readout hydrogen peroxide biological indicator.

In general, a sterilization process includes placing the biological indicator of the present disclosure in a sterilizer. In some embodiments, the sterilizer includes a sterilization chamber that can be sized to accommodate a plurality of articles to be sterilized and can be equipped with a means of evacuating air and/or other gases from the chamber and a means for adding a sterilant to the chamber. The self-contained biological indicator can be positioned in areas of the sterilizer that are most difficult to sterilize. Alternately, the biological indicator can be positioned in process challenge devices to simulate sterilization conditions where sterilant may not be delivered as directly as would be the case in more favorable sterilization circumstances.

The sterilant can be added to the sterilization chamber after evacuating the chamber of at least a portion of any air or other gas present in the chamber. Alternatively, sterilant can be added to the chamber without evacuating the chamber. A series of evacuation steps can be used to assure that the sterilant reaches all desired areas within the chamber and contacts all desired article(s) to be sterilized, including the biological indicator.

The self-contained biological indicators are capable of determining the efficacy of one or more steam sterilization cycles chosen from the powerset of the following eleven cycles:121° C. gravity, 121° C. pre-vac, 121° C. SFPP, 132° C. gravity, 132° C. pre-vac, 132° C. SFPP, 134° C. pre-vac, 134° C. SFPP, 135° C. gravity, 135° C. pre-vac, and 135° C. SFPP, preferably within less than 1 hr.

### Detection of Enzymatic Activity and Determination of a Successful Sterilization Process

In another aspect, the present disclosure provides a method for determining the efficacy of a sterilization process. In any embodiment, the method comprises exposing a plurality of test microorganisms that are disposed in a housing to the sterilization process. Suitable test microorganisms can be any test microorganism described herein. In some embodiments, the test microorganisms can be disposed in any embodiment of a sterilization process indicator disclosed herein. Alternatively, the test microorganisms can be disposed in a container (e.g., a tube) or on a substrate (e.g., a paper strip, a glass slide, or yarn). Exposing the test microorganisms to the sterilization process comprises placing the article on which (or in which) the test microorganisms is disposed into a vessel (e.g., an automated sterilizer) in which the sterilization process is conducted.

In any embodiment of the method, the test microorganisms comprise and/or is capable of producing an enzyme capable of reacting with an enzyme substrate to produce a :fluorescent product as described herein.

After exposing the test microorganisms to the sterilization process, the method comprises bringing the plurality of test microorganisms into contact with a liquid composition. The liquid composition can be any suitable liquid composition (e.g., an aqueous liquid composition) according to the present disclosure. Bringing the plurality of test microorganisms into contact with the liquid composition comprises placing the test microorganisms in liquid contact with the enzyme substrate.

After bringing the plurality of test microorganisms into contact with the liquid composition, a resulting mixture of the plurality of test microorganisms and the liquid composition further comprises a nutrient composition, the enzyme substrate, and a salt compound. The nutrient composition, the enzyme substrate, and the salt compound can be any suitable nutrient composition, enzyme substrate, and salt compound described herein. In certain embodiments, any one or all of the nutrient composition, the enzyme substrate, and the salt compound can be disposed in the liquid composition prior to forming the mixture with the test microorganisms.

In certain embodiments wherein the test microorganisms are disposed in a container (e.g., a tube), placing the test microorganisms in liquid contact with the enzyme substrate can comprise adding (e.g., by pipet) the liquid composition to the container containing the test microorganisms. Optionally, prior to pipetting the liquid composition into the container, the liquid composition comprises any one or all of the nutrient composition, the enzyme substrate, and the salt compound as described herein. In certain embodiments wherein the test microorganisms are disposed in a self-contained sterilization process indicator according to the present disclosure, placing the test microorganisms in liquid contact with the enzyme substrate can comprise actuating (e.g., crushing or otherwise opening) the container containing the liquid composition to release the liquid composition as described herein. Optionally, after placing the test microorganisms in liquid contact with the enzyme substrate, the components can be mixed (e.g., by manual or mechanical agitation or vortex action).

After bringing the test microorganisms into contact with the liquid composition, a resulting mixture of the plurality of test microorganisms and the liquid composition comprises a nutrient composition, the enzyme substrate, and a salt compound; each as described herein. The nutrient composition facilitates germination and/or outgrowth of the test microorganisms. In certain embodiments, the salt compound is present in the mixture at a concentration described herein (e.g., at least 5 mM and up to 50 mM; with the proviso that when the concentration equals 10 mM, the salt compound is not potassium phosphate).

After bringing the test microorganisms into contact with the liquid composition, the method comprises incubating the mixture for a period of time. Incubating the mixture for a period of time comprises incubating the mixture at a specified temperature. The specified temperature can be any suitable incubation temperature described herein for the test microorganism and/or the enzyme activity. The period of time can be any suitable period of time of incubation described herein. In certain embodiments, the specified period of time is less than 8 hours, in some embodiments, less than 1 hour, in some embodiments, less than 30 minutes, in some embodiments, less than 15 minutes, in some embodiments, less than 5 minutes, and in some embodiments, less than 1 minute. In other embodiments, the suitable incubation time for the biological indicator of this disclosure is from 10 min to 1 hr, or from 10 min to 50 min, or from 10-30 min, or from 10-20 min, or from 10-25 min, or from 15 to 30 min, or from 15-25 min, or from 15-20 min.

During and/or after incubating the mixture for a period of time, the method comprises detecting the fluorescent product formed in the mixture. Detecting the fluorescent product comprises directing a first electromagnetic radiation (e.g., radiation within the ultraviolet spectrum of electromagnetic energy) into the mixture and detecting a second electromagnetic radiation (e.g., radiation within the ultraviolet spectrum or visible spectrum of electromagnetic energy) emitted by the fluorescent product in the mixture, as described herein. In certain embodiments, detecting electromagnetic radiation emitted by the fluorescent product comprises detecting electromagnetic radiation using an automated detector (e.g., an auto-reader as described herein).

In any embodiment, detecting the fluorescent product comprises detecting a quantity of fluorescence emitted by the fluorescent product. In any embodiment, the quantity of fluorescence detected can be compared to a threshold quantity. In any embodiment, a first quantity of fluorescence detected after a first specified time period can be compared to a second quantity of fluorescence detected after a second specified time period. In certain embodiments, detecting at least a threshold quantity of the fluorescent product indicates a lack of efficacy of the sterilization process (i.e., not all of the plurality of test microorganisms and/or the enzyme activity associated therewith were inactivated (e.g., killed) by the sterilization process).

In certain embodiments, a method according to the present disclosure can be used to determining efficacy of a sterilization process that uses a sterilant selected from the group consisting of steam, ethylene oxide gas, hydrogen peroxide vapor, methyl bromide, chlorine dioxide, fomlaldehyde, peracetic acid, ozone, ionizing radiation, and a combination of any two or more of the foregoing sterilants.

As mentioned in the previous section, after the indicator is exposed to the sterilization process, the test microorganisms can be incubated in the nutrient medium to determine whether any of the test microorganisms survived the sterilization process, with microorganism growth indicating that the sterilization process was insufficient to destroy all of the test microorganisms.

In some embodiments, the cap of the biological indicator can be coupled to the body of the biological indicator during sterilization in a first position that maintains fluid communication between the interior of the biological indicator and ambience, allowing the sterilant to reach the interior of the biological indicator. After sterilization, in order to activate the biological indicator, the cap can be pressed further onto the tube (e.g., to a second position in which the interior of the biological indicator is no longer in fluid communication with ambience) to maintain sterility and reduce the evaporation rate of the liquid composition. As mentioned previously, the liquid composition is maintained separate from the test microorganisms in the frangible container during sterilization but is released into the interior of the housing after sterilization as part of the activation by fracturing, puncturing, piercing, crushing, cracking, breaking, or the like, the frangible container.

In some embodiments of the present disclosure, closing the biological indicator (e.g., moving a portion of the biological indicator, such as the cap, relative to another portion to seal the interior) can include or cause fracturing, puncturing, etc. of the frangible container containing the liquid composition, such that closing the biological indicator causes activation of the biological indicator.

After activation, the mixture resulting from placing the liquid composition in contact with the test microorganisms is incubated is continued for a period of time and under conditions that would be sufficient to liberate a detectable amount of the enzyme modified product, assuming, of course, that any of the test microorganisms remains active. In general, the amount of product which is detectable by known methods is at least 10⁻⁸ molar. Preferably, the incubation conditions are sufficient to generate at least 10⁻⁸ molar of fluorescently-detectable compound, more preferably, about 10⁻⁶ molar to 10⁻⁵ molar of fluorescently-detectable compound. The incubation time and temperature needed to produce a detectable amount of fluorescently-detectable compound will depend upon the identity of the enzyme and the substrate, and the concentrations of each present in the reaction mixture. In general, the incubation time required is between about 1 minute and 12 hours, and the incubation is between about 20° C. and 70° C. Preferably, where Bacillus subtilis or Geobacillus stearothermophilus is the source of the enzyme, the incubation time is from about 10 minutes and 3 hours, or from 10 minutes to 1 hour, or from 15 minutes and 1 hour, or from 15 minutes and 30 minutes, or from 15 minutes to 25 minutes, and the incubation temperature is from about 30° C. to about 40° C., and from about 52° C. to 65° C., respectively.

To detect a detectable change in the test microorganisms the biological indicator can be assayed immediately after the liquid composition and the test microorganisms have been combined to achieve a baseline reading. After that, any detectable change from the baseline reading can be detected. The biological indicator can be monitored and measured continuously or intermittently. In some embodiments, a portion of, or the entire, incubating step may be carried out prior to measuring the detectable change. In some embodiments, incubation can be carried out at one temperature (e.g., at 37° C., at 50-60° C., etc.), and measuring of the detectable change can be carried out at a different temperature (e.g., at room temperature, 25° C., or at 37° C.). In other embodiments, the incubation and measurement of fluorescence occurs at the same temperature.

The readout time of the biological indicator (i.e., the time to determine the effectiveness of the sterilization process) can be, in some embodiments, less than 8 hours, in some embodiments, less than 1 hour, in some embodiments, less than 30 minutes, in some embodiments, less than 15 minutes, in some embodiments, less than 5 minutes, and in some embodiments, less than 1 minute. In other embodiments, the readout time for the biological indicator of this disclosure is from 10 min to 1 hr, or from 10 min to 50 min, or from 10-30 min, or from 10-20 min, or from 10-25 min, or from 15 to 30 min, or from 15-25 min, or from 15-20 min. The detection of fluorescence above the baseline reading that would indicate presence of viable test microorganisms (i.e., a failed sterilization process) can be performed according to any method know in the art, including area under curve (in a plot of time vs fluorescence intensity), monitoring a change in slope of the curve, using a threshold value for the fluorescence, etc., or a combination thereof of two or more techniques.

One of the advantages of the biological indicators of this disclosure is that a single type can be used for various sterilization conditions. The working examples below show a single type of biological indicator can be used for all of the following steam sterilization cycles: 121° C. gravity, 121° C. pre-vac, 121° C. SFPP, 132° C. gravity, 132° C. pre-vac, 132° C. SFPP, 134° C. pre-vac, 134° C. SC FPP, 135° C. gravity, 135° C. pre-vac, and 135° C. SFPP. For that reason, the biological indicator can be used for any subset of cycles chosen from the set above. That is, a single biological indicator is capable of determining the efficacy of one or more sterilization cycles chosen from the powerset of 121° C. gravity, 121° C. pre-vac, 121° C. SFPP, 132° C. gravity, 132° C. pre-vac, 132° C. SFPP, 134° C. pre-vac, 134° C. SFPP, 135° C. gravity, 135° C. pre-vac, and 135° C. SFPP.

In addition to being able to determine the efficacy of any of the above sterilization cycles, the biological indicator is capable of doing so in less than one hour. In fact, in some embodiments, the biological indicator has a readout time of less than 1 hour, in some embodiments, less than 30 minutes, in some embodiments, less than 15 minutes, in other embodiments, the readout time is from 10 min to 1 hr, or from 10 min to 50 min, or from 10-30 min, or from 10-20 min, or from 10-25 min, or from 15 to 30 min, or from 15-25 min, or from 15-20 min.

### Kits

In another aspect, the present disclosure provides a kit that can be used for determining the efficacy of a sterilization process. In one embodiment, the kit comprises i) a plurality of test microorganisms comprising and/or capable of producing an enzyme capable of catalyzing the cleavage of an enzyme substrate, ii) the enzyme substrate, iii) a liquid composition comprising the enzyme substrate, and iv) an effective amount of a salt compound; wherein the salt compound, when dissolved in the liquid composition, is present at a concentration of at least 0.5 mM and up to 50 mM of the salt compound in the liquid composition; with the proviso that when the concentration equals 10 mM, the salt compound is not potassium phosphate. A product of the cleavage of the enzyme substrate by the enzyme can be detected by its fluorescence.

In another embodiment, the kit comprises any embodiment of the self-contained biological indicator of the present disclosure.

### EXAMPLES

### Example 1: Biological Indicator with Added Salt Compound in a Steam Sterilization Process

Type I Biological Indicators (Salt Compound Added to the BI with the Spores).

Components from commercial 1492V biological indicators (3M Company, St. Paul, Minn.) were used to assemble the biological indicators of these Examples. Spores of Geobacillus stearothermophilus were produced in liquid sporulation medium. The spores were washed in sterile deionized water and were resuspended in sterile phosphate buffer solutions (pH 7.2). The concentration of phosphate buffer in the solutions was adjusted so that, when the spores were resuspended in the nutrient medium (approximately 0.5 mL) released from the ampules during activation of the biological indicators, the concentration of the phosphate buffer in the nutrient medium became one of the concentrations listed in Tables 1 and 2. Aliquots of the spore suspensions were pipetted onto a polypropylene film carrier (approximately 0.065 mm thick and 5 mm diameter) with a spore population of approximately 1×10⁶ colony forming units per carrier (CFU/carrier). The spore carriers of the commercial biological indicators were replaced with the carriers described herein and the biological indicators were assembled as shown in U.S. Pat. No. 10,047,334.

Type II Biological Indicators (Salt Compound Added to the BI with the Nutrient Medium).

Components from commercial 1492V biological indicators (3M Company, St. Paul, Minn.) were used to assemble the biological indicators of these Examples with the exception that the ampules of nutrient medium in the commercial biological indicators were replaced with ampules of sterile medium containing peptones, amino acids, a fermentable carbohydrate, bromocresol purple, 300 mg/L 4-methylumbelliferyl-α-D-glucopyranoside and a potassium phosphate buffer (pH 7.2) at one of the concentrations designated in Table 2. The medium was suitable for growth and detection of Geobacillus stearothermophilus. Spores of Geobacillus stearothermophilus were produced in liquid sporulation medium. The spores were washed and resuspended in sterile deionized water. Aliquots of the spore suspensions were pipetted onto a polypropylene film carrier (approximately 0.065 mm thick and 5 mm diameter) with a spore population of approximately 1×10⁶ colony forming units per carrier (CFU/carrier). The spore carriers of the commercial biological indicators were replaced with the carriers described herein and the biological indicators were assembled as shown in U.S. Pat. No. 10,047,334.

Five assembled Types I and II self-contained biological indicators from Example 1 were placed in a steam resistometer (Model 101; H & W Technology; Rochester, N.Y.), where they were exposed to a steam 121° C. pre-vacuum sterilization cycle. After the biological indicators were removed from the sterilizer, they were activated by crushing the ampule of liquid medium according to the manufacturer's instructions and the biological indicators were incubated at 60° C. for 24 minutes using an autoreader (Model 490H) available from 3M Company (St. Paul, Minn.). The biological indicators were then transferred into an incubator for approximately 7 days for an indication of spore germination and growth (i.e., a change in the color of the growth medium from purple to yellow). The percentage of fluorescence positive is shown in Table 1. The effect of the potassium phosphate buffer on the growth-positive biological indicators is shown in Table 2.

TABLE 1
Effect of potassium phosphate buffer ionic strength on the percentage
of fluorescence positive result. Each reported data point is a percentage
of five Type I self-contained individual biological indicators.

| | |
|---|---|
| Potassium Phosphate | Steam Exposure |

| concentration | 5.5 min | 6 min | 6.5 min | 7 min |
|---|---|---|---|---|
| 0.000M | 100% | 80% | 40% | 0% |
| 0.001M | 100% | 80% | 60% | 60% |
| 0.005M | 100% | 100% | 80% | 80% |
| 0.025M | 100% | 100% | 100% | 100% |
| 0.050M | 100% | 100% | 100% | 100% |

TABLE 2
Effect of potassium phosphate buffer ionic strength on percentage
of growth positive result. Each reported data point is a percentage
of five Type II self-contained individual biological indicators.

| | |
|---|---|
| Potassium Phosphate | Steam Exposure |

| concentration | 3 min | 6 min |
|---|---|---|
| 0.000M | 100% | 80% |
| 0.001M | 100% | 60% |
| 0.005M | 100% | 60% |
| 0.010M | 80% | 20% |
| 0.025M | 0% | 0% |

The data indicate a higher percentage of florescence positive associated with a lower percentage of survival at the highest concentration of the potassium phosphate.

### Examples 2-5: Use of Biological Indicator with Added Salt Compound in a Hydrogen Peroxide Sterilization Process

Type I Biological Indicators (Salt Compound Added to the BI with the Spores).

Components from commercial 1295 biological indicators (3M Company, St. Paul, Minn.) were used to assemble the biological indicators of these Examples. Spores of Geobacillus stearothermophilus were produced in liquid sporulation medium. The spores were washed in sterile deionized water and were resuspended in sterile phosphate buffer solutions (pH 7.2). The concentration of phosphate buffer in the solutions was adjusted so that, when the spores were resuspended in the nutrient medium (approximately 0.6 mL) released from the ampules during activation of the Biological indicators, the concentration of the phosphate buffer in the nutrient medium became one of the concentrations listed in Tables 3 and 4. Aliquots of the spore suspensions were pipetted onto a polypropylene film carrier (approximately 0.065 mm thick and 5 mm diameter) with a spore population of approximately 1×10⁷ colony forming units per carrier (CFU/carrier). The spore carriers of the commercial biological indicators were replaced with the carriers described herein and the biological indicators were assembled as shown in U.S. Pat. No. 10,047,334.

Type II Biological Indicators (Salt Compound Added to the BI with the Nutrient Medium).

Components from commercial 1295 biological indicators (3M Company, St. Paul, Minn.) were used to assemble the biological indicators of these Examples with the exception that the ampules of nutrient medium in the commercial biological indicators were replaced with ampules of sterile medium containing peptones, amino acids a fermentable carbohydrate, bromocresol purple, 300 mg/L 4-methylumbelliferyl-α-D-glucopyranoside and a potassium phosphate buffer (pH 7.2) at one of the concentrations designated in Tables 3 and 4. The medium was suitable for growth and detection of Geobacillus stearothermophilus. Spores of Geobacillus stearothermophilus were produced in liquid sporulation medium. The spores were washed and resuspended in sterile deionized water. Aliquots of the spore suspensions were pipetted onto a polypropylene film carrier (approximately 0.065 mm thick and 5 mm diameter) with a spore population of approximately 1×10⁷ colony forming units per carrier (CFU/carrier). The spore carriers of the commercial biological indicators were replaced with the carriers described herein and the biological indicators were assembled as shown in U.S. Pat. No. 10,047,334.

The assembled Type I self-contained biological indicators were placed in a hydrogen peroxide resistometer vessel (The Sterilucent™ PSD-85 Hydrogen Peroxide Sterilizer (Sterilucent Inc., Minneapolis, Minn.) where they were exposed to a various lengths of time (1 second, 5 seconds, 10 seconds, and 15 seconds as shown in Table 3) of 59% vaporized aqueous solution of hydrogen peroxide at a temperature of 55° C. and a pressure of 0.05 kPa. After the biological indicators were removed from the resistometer, they were activated by crushing the ampule of liquid medium and the fluorescence was measured using an autoreader (Model 490H) available from 3M Company (St. Paul, Minn.). The time (in minutes) at which a positive fluorescence was detected by the autoreader was recorded. The data are shown in Table 3. The data show that the time to turn fluorescence positive is inversely related to the concentration of the potassium phosphate buffer regardless of the hydrogen peroxide exposure time.

TABLE 3
Effect of potassium phosphate buffer ionic strength on the fluorescence
time to positive result. The data report the average time (in
minutes) for the biological indicators to turn fluorescent-
positive. Each reported data point is an average of five Type
I self-contained individual biological indicators.

| | Hydrogen Peroxide Exposure | | | |
|---|---|---|---|---|
| Potassium Phosphate | 1 | 5 | 10 | 15 |
| concentration | second | seconds | seconds | seconds |
| 0.000M | 18 | 19 | 16 | 19 |
| 0.001M | 14 | 15 | 16 | 18 |
| 0.005M | 10 | 10 | 10 | 10 |
| 0.010M | 10 | 11 | 10 | 13 |
| 0.050M | 10 | 11 | 10 | 12 |

The data indicate a faster fluorescence time to positive result at the highest concentration of the potassium phosphate.

### Examples 6-9: Use of Biological Indicator with Added Salt Compound in a Hydrogen Peroxide Sterilization Process

Forty assembled Type II self-contained biological indicators were placed in a hydrogen peroxide resistometer vessel (The Sterilucent™ PSD-85 Hydrogen Peroxide Sterilizer (Sterilucent Inc., Minneapolis, Minn.) where they were exposed to a various lengths of time (1 second, 5 seconds, 10 seconds, and 15 seconds as shown in Table 3) of 59% vaporized aqueous solution of hydrogen peroxide at a temperature of 55° C. and a pressure of 0.05 kPa. After the biological indicators were removed from the sterilizer, they were activated by crushing the ampule of liquid medium according to the manufacturer's instructions and the biological indicators were incubated at 60° C. for approximately 7 days. The biological indicators were then observed for an indication of spore germination and growth (i.e., a change in the color of the growth medium from purple to yellow). The percentage of growth-positive biological indicators is shown in Table 4.

TABLE 4
Effect of potassium phosphate buffer ionic strength on the
growth-positive after exposure to hydrogen peroxide sterilant.
Each reported data point is a percentage of forty Type II
self-contained individual biological indicators.

| | |
|---|---|
| Potassium Phosphate | Hydrogen Peroxide Exposure |

| concentration | 1 second | 30 seconds |
|---|---|---|
| 0.000M | 100% | 25% |
| 0.001M | 100% | 37.5% |
| 0.005M | 40% | 37.5% |
| 0.010M | 100% | 12.5% |
| 0.050M | 0% | 0% |

The data indicate a lower percentage of survival (as indicated by growth) at the highest concentration of the potassium phosphate.

### Examples 10-12. Effect of Salt Compound on the Detection of Spore Enzyme Activity

A liquid composition consisting of growth media comprising 0.1 mg of 4-methylumbelliferone (4-MU) in 1 mL of deionized water containing a mixture of peptones, amino acids, a fermentable carbohydrate, and bromocresol purple to facilitate growth and detection of Geobacillus stearothermophilus was placed into containers. In the Control I and Control II, the liquid composition pH was adjusted to a value of 7 and 8 respectively using HCl or NaOH, as needed. In Example 10, the sample solution additionally contained the salt compound KH₂PO₄/K₂HPO₄ to yield a final pH value of 8 and concentration of 0.025 M. In Example 11, the sample solution additionally contained the salt compound (NH₄)₂CO₃ to yield a final pH value of 8 and a concentration of 0.01M. In Example 12, the sample solution additionally contained Tris-HCl to yield a final pH value of 8 and a concentration of 0.01M. The aqueous solutions giving bound values of pH at 25° C. using these salts were obtained according the standard procedure described by CRC Handbook of Chemistry and Physics (56th edition, page D-134). (Unless otherwise noted, all chemical used in the preparation of these samples are available from available from Sigma-Aldrich, St. Louis, Mo.).

Geobacillus stearothermophilus spore crops were produced as described in Example 1. After harvesting, spore crops were washed by centrifugation and suspended in deionized water (shown and described in U.S. Pat. No. 10,047,334; which is incorporated herein by reference in its entirety). The spore suspension (approximately 10⁸ CFU/mL) was added to each container holding the sample solutions described above to yield a final sample spore concentration of approximately 1×10⁶ colony forming units per milliliter in the liquid composition.

Fluorescence spectra of these sample solutions were collected using a Fluoromax-4 spectrofluorometer made by HORIBA JOBIN YVON (available from HORIBA Scientific, Edison, N.J.). The excitation wavelength used was 360 nm. The fluorescence intensity was measured at an emission wavelength of 450 nm. Fluorescence Intensity in Relative Fluorescence Units (RFUs) was measured kinetically from each of the sample solutions as a function of time in 100 seconds increments for a total of 2 hours.

Fluorescent intensity measurements (as a percentage of the control) reported in Table 5 are an average of three replicates. One standard deviation for all fluorescent intensity measurements reported in the table is ±10% of the measured fluorescent intensity. Vmax represents the slope characterizing the linear increase in the fluorescent signal with time for the first 10 minutes of the assay.

For a constant pH of the growth media solution, the introduction of salt creates a measurable effect in Vmax, fluorescent intensity as well as in how stable that fluorescent intensity remains over extended time. Depending on the salt system the effect can be to increase or decrease both Vmax and the fluorescent intensity. Addition of (NH₄)₂CO₃ does not have an impact of the activity of the enzyme but does result in better stability of the fluorescent signal over extended time in comparison to the control system (no salt). Adding KH₂PO₄/K₂HPO₄ increases the enzyme's activity substantially and provides a fluorescent intensity that is stable for up to 2 hours after initiating the kinetic assay. Using a Tris salt results in significant inhibition of the enzymatic activity.

TABLE 5
Results of Kinetic Assay for B. stearothermophilus α-glucosidase Enzymatic Activity.

| | | | | Fluorescence | | Fluorescence | Fluorescence |
|---|---|---|---|---|---|---|---|
| | | | | Intensity at | | Intensity at | Intensity at |
| | Media | | Vmax | 24 minutes | | 60 minutes | 120 minutes |
| Example | pH | Salt Compound | (RFUs/sec) | (% of control) | | (RFUs) | (RFUs) |
| Control I | 7 | None | 160 ± 23 | 131034 | (100%) | 146938 | 93877 |
| Control II | 8 | None | 255 ± 54 | 176865 | (135%) | 155102 | 44897 |
| 10 | 8 | KH₂PO₄/K₂HPO₄ | 343 ± 67 | 224137 | (171%) | 224489 | 226530 |
| 11 | 8 | (NH₄)₂CO₃ | 255 ± 54 | 178721 | (136%) | 177551 | 81632 |
| 12 | 8 | NH₂C(CH₂OH)₃Cl | 9 ± 3 | 18966 | (14%) | 65306 | 89796 |
| | | [Tris-HCl] | | | | | |

A liquid composition similar to the one used in Examples 10-12 was used to prepare containers holding the solutions for Examples 13-15. In the Control, the solution pH was adjusted to a value of 7 using HCl or NaOH, as needed. In Example 13, the solution additionally contained the buffer salt system KH₂PO₄/K₂HPO₄ to yield a final pH value of 8 and concentration of 025 M. In Example 14, the sample solution additionally contained (NH₄)₂CO₃ to yield a final pH value of 8 and a concentration of 0.01M. In Example 15, the sample solution additionally contained Tris-HCl to yield a final pH value of 8 and a concentration of 0.01M. The aqueous solutions giving bound values of pH at 25° C. using these salts were obtained according the standard procedure described by CRC Handbook of Chemistry and Physics (56th edition, page D-134). (Unless otherwise noted, all chemical used in the preparation of these samples are available from available from Sigma-Aldrich, St. Louis, Mo.).

Fluorescence spectra of these aqueous 4-MU solutions were collected using a Fluoromax-4 spectrofluorometer made by HORIBA JOBIN YVON (available from HORIBA Scientific, Edison, N.J.). The excitation wavelength used was 360 nm. The fluorescence intensity was measured at an emission wavelength of 450 nm. The data are shown in Table 6.

The presence of salt has no effect of the fluorescence intensity measured from the 4-MU substrate, whereas pH has a very significant effect of the intensity of 4-MU. For an excitation wavelength of 360 nm, the fluorescent intensity measured at the emission wavelength of 450 nm is on average 4.5 times greater when the media solution is at a pH=8 in comparison to a pH=7.

TABLE 6
Effect of salt on 4-MU fluorescence.

| | | | Fluorescence | Ratio of |
|---|---|---|---|---|
| | | | Intensity | Fluorescent |
| | Media | Media Salt | at 450 nm | Intensity |
| Example | pH | System | (RFUs) | to Control |
| Control | 7 | None | 114177 ± 1412 | 1.0 |
| 13 | 8 | KH₂PO₄/K₂HPO₄ | 513634 ± 3776 | 4.5 |
| 14 | 8 | (NH₄)₂CO₃ | 538002 ± 1137 | 4.7 |
| 15 | 8 | NH₂C(CH₂OH)₃ | 496782 ± 5251 | 4.4 |
| | | [Tris-HCl] | | |

1. A self-contained biological indicator comprising:
a housing, the housing containing:
a plurality of test microorganisms comprising and/or capable of producing an enzyme capable of catalyzing a cleavage of an enzyme substrate;
the enzyme substrate;
a nutrient composition, wherein the nutrient composition facilitates germination and/or outgrowth of the test microorganisms;
a container containing a liquid composition, wherein the container is adapted to allow selective fluid communication between the liquid composition and the test microorganisms; and
an effective amount of a salt compound; wherein the salt compound is mixed with the plurality of test microorganisms, and when the salt compound is dissolved in the liquid composition, the salt compound is present at a concentration of at least 0.5 mM and up to 50 mM in the liquid composition; with the proviso that when the concentration equals 10 mM, the salt compound is not potassium phosphate;
wherein the cleavage of the enzyme substrate by the enzyme produces a fluorescently detectable compound.

2. The self-contained biological indicator of claim 1, wherein the salt compound is selected from the group consisting of a salt of any ion selected from the group consisting of acetate; borate; citrate; carbonate; bicarbonate; phosphate; hydrogen phosphate; dihydrogen phosphate; chloride; sulfate; N,N-Bis(2-hydroxyethyl)-2-aminoethanesulfonate; N,N-Bis(2-hydroxyethyl)glycine; 3-(Cyclohexylamino)-2-hydroxy-1-propanesulfonic acid; N-Cyclohexyl-2-aminoethanesulfonate; imidazolium; 2-(N-Morpholino)ethanesulfonate; 3-(N-morpholino)propanesulfonic acid; tricine, 2-Amino-2-(hydroxymethyl)propane-1,3-diol; and a combination of any two or more of the foregoing salts.

3. The self-contained biological indicator of claim 1, wherein the enzyme is selected from the group consisting of α-glucosidase, α-galactosidase, lipase, esterase, acid phosphatase, alkaline phosphatase, protease, aminopeptidase, chymotrypsin, β-glucosidase, β-galactosidase, α-glucoronidase, β-glucoronidase, phosphohydrolase, calpain, α-mannosidase, β-mannosidase, α-L-fucosidase, leucine aminopeptidase, α-L-arabinofuranosidase, cysteine aminopeptidase, valine aminopeptidase, β-xylosidase, glucanase, cellobiosidase, cellulase, α-arabinosidase, glycanase, sulfatase, butyrase, glycosidase, arabinosidase, and a combination of any two or more of the foregoing enzymes.

4. The self-contained biological indicator of claim 1, wherein the enzyme substrate comprises a derivative of 4-methylumbelliferone or a derivative of 7-amino-4-methylcoumarin.

5. The self-contained biological indicator of claim 1, wherein the enzyme comprises α-D-glucosidase, wherein the enzyme substrate comprises 4-methylumbelliferyl-α-D-glucopyranoside.

6. A kit, comprising:
a housing;
a plurality of test microorganisms comprising and/or capable of producing an enzyme capable of catalyzing the cleavage of an enzyme substrate;
a nutrient composition, wherein the nutrient composition facilitates germination and/or outgrowth of the test microorganisms;
the enzyme substrate, wherein the enzyme substrate comprises a fluorescently detectable component;
a liquid composition; and
an effective amount of a salt compound; wherein the salt compound is mixed with the plurality of test microorganisms, and when the salt compound is dissolved in the liquid composition, the salt compound is present at a concentration of at least 0.5 mM and up to 50 mM in the liquid composition; with the proviso that when the concentration equals 10 mM, the salt compound is not potassium phosphate.

7. The kit of claim 6, wherein one or more of the nutrient composition, the enzyme substrate, the liquid composition, the salt compound, and the plurality of test microorganisms is disposed in the housing.

8. The kit of claim 6, wherein the liquid composition is disposed in a frangible container.

9. A kit comprising the self-contained biological indicator of claim 1.

10. A method of determining efficacy of a sterilization process, the method comprising:
exposing a mixture of a salt compound and a plurality of test microorganisms that is disposed in a housing to the sterilization process;
wherein the plurality of test microorganisms comprises and/or is capable of producing an enzyme capable of reacting with an enzyme substrate to produce a fluorescent product;
after exposing the test microorganisms to the sterilization process, bringing the mixture into contact with a liquid composition;
wherein bringing the mixture into contact with the liquid composition comprises placing the mixture in liquid contact with the fluorogenic enzyme substrate;
wherein, after bringing the mixture into contact with the liquid composition, a resulting second mixture of the plurality of test microorganisms and the liquid composition comprises a nutrient composition, the enzyme substrate, and the salt compound;
wherein the salt compound is present in the second mixture at a concentration of at least 0.5 and up to 50 mM; with the proviso that when the concentration equals 10 mM, the salt compound is not potassium phosphate
wherein the nutrient composition facilitates germination and/or outgrowth of the test microorganisms;
incubating the second mixture for a period of time; and
detecting the fluorescent product in the second mixture;
wherein detecting at least a threshold quantity of the fluorescent product indicates a lack of efficacy of the sterilization process.

11. The method of claim 10, wherein incubating the second mixture for a period of time comprises incubating the second mixture at a specified temperature.

12. The method of claim 10, wherein the period of time is a specified period of time, wherein the specified period of time is less than or equal to 180 minutes, wherein detecting less than a threshold quantity of the fluorescent product after the specified period of time indicates efficacy of the sterilization process.

13. The method of claim 12, wherein the specified period of time is less than or equal to 180 minutes.

14. The method of claim 10, wherein the enzyme is selected from the group consisting of α-glucosidase, α-galactosidase, lipase, esterase, acid phosphatase, alkaline phosphatase, proteases, aminopeptidase, chymotrypsin, β-glucosidase, β-galactosidase, α-glucoronidase, β-glucoronidase, phosphohydrolase, plasmin, thrombin, trypsin, calpain, α-mannosidase, β-mannosidase, α-L-fucosidase, leucine aminopeptidase, α-L-arabinofuranosidase, cysteine aminopeptidase, valine aminopeptidase, β-xylosidase, α-L-iduronidase, glucanase, cellobiosidase, cellulase, α-arabinosidase, glycanase, sulfatase, butyrase, glycosidase, arabinoside, and a combination of any two or more of the foregoing enzymes.

15. The method of claim 10, wherein the test microorganisms are spores produced by a microorganism selected from the group consisting of Geobacillus stearothermophilus, Bacillus stearothermophilus, Bacillus subtilis, Bacillus atrophaeus, Bacillus megaterium, Bacillus coagulans, Clostridium sporogenes, Bacillus pumilus, or a combination of any two or more of the foregoing microorganisms.

16. The method of claim 10, wherein detecting the fluorescent product comprises quantifying fluorescence emitted by the fluorescent product.

17. The method of claim 10, wherein the sterilization process is a process using a sterilant selected from the group consisting of steam, ethylene oxide gas, hydrogen peroxide vapor, methyl bromide, chlorine dioxide, formaldehyde, peracetic acid, ozone, ionizing radiation, and a combination of any two or more of the foregoing sterilants.

18. A system, comprising:
the self-contained biological indicator of claim 1; and
an automated reader configured to:
receive at least a portion of the biological indicator;
direct a first wavelength of electromagnetic radiation into the liquid composition in the housing; and
detect or measure a quantity of a second wavelength of electromagnetic radiation emitted by the fluorescent product.

19. The system of claim 18, wherein the self-contained biological indicator is adapted to be used to determine efficacy of any steam sterilization process selected from the group consisting of 121° C. gravity process, 121° C. pre-vac process, 121° C. SFPP process, 132° C. gravity process, 132° C. pre-vac process, 132° C. SFPP process, 134° C. pre-vac process, 134° C. SFPP process, 135° C. gravity process, 135° C. pre-vac process, and 135° C. SFPP process.
